(19) United States
(12) Patent Application Publication
(10) Pub. No.: US 2024/0130644 A1
(43) Pub. Date: Apr. 25, 2024

(54) TRANSCUTANEOUS MEDICAL DEVICE WITH VARIABLE STIFFNESS

(71) Applicant: DexCom, Inc., San Diego, CA (US)

(72) Inventors: Mark C. Brister, San Diego, CA (US); James H. Brauker, San Diego, CA (US)

(21) Appl. No.: 18/402,635

(22) Filed: Jan. 2, 2024

Related U.S. Application Data
(63) Continuation of application No. 16/942,528, filed on Jul. 29, 2020.
Application No. 16/942,528 is a continuation of application No. 16/007,725, filed on Jun. 13, 2018, now abandoned.
Application No. 16/007,725 is a continuation of application No. 14/266,408, filed on Apr. 30, 2014, now abandoned.
Application No. 14/266,408 is a continuation of application No. 12/105,227, filed on Apr. 17, 2008, now Pat. No. 8,812,072.
Application No. 12/105,227 is a continuation of application No. 11/077,759, filed on Mar. 10, 2005, now Pat. No. 7,783,333.
(60) Provisional application No. 60/587,800, filed on Jul. 13, 2004.

(51) Int. Cl. A61B 5/145; A61B 5/00; A61B 5/1468; A61B 5/1486; A61B 5/15

(52) U.S. Cl. CPC A61B 5/14532; A61B 5/1411; A61B 5/14546; A61B 5/1468; A61B 5/14865; A61B 5/6833; A61B 5/150022

(57) ABSTRACT

The present invention relates generally to variable stiffness transcutaneous medical devices including a distal portion designed to be more flexible than a proximal portion. The variable stiffness can be provided by a variable pitch in one or more wires of the device, a variable cross-section in one or more wires of the device, and/or a variable hardening and/or softening in one or more wires of the device.

### INCORPORATION BY REFERENCE TO RELATED APPLICATIONS

Any and all priority claims identified in the Application Data Sheet, or any correction thereto, are hereby incorporated by reference under 37 CFR 1.57. This application is a continuation of U.S. application Ser. No. 16/942,528, filed Jul. 29, 2020, which is a continuation of U.S. application Ser. No. 16/007,725, filed Jun. 13, 2018, now abandoned, which is a continuation of U.S. application Ser. No. 14/266,408, filed Apr. 30, 2014, now abandoned, which is a continuation of U.S. application Ser. No. 12/105,227, filed Apr. 17, 2008, now U.S. Pat. No. 8,812,072, which is a continuation of U.S. application Ser. No. 11/077,759, filed Mar. 10, 2005, now U.S. Pat. No. 7,783,333, which claims the benefit of U.S. Provisional Application No. 60/587,800 filed Jul. 13, 2004. Each of the aforementioned applications is incorporated by reference herein in its entirety, and each is hereby expressly made a part of this specification.

### FIELD OF THE INVENTION

The present invention relates generally to systems and methods for use with partially implantable medical devices. More particularly, the present invention relates to systems and methods for use with transcutaneous analyte sensors.

### BACKGROUND OF THE INVENTION

Transcutaneous medical devices are useful in medicine for providing the enhanced functionality of a wholly implantable medical device while avoiding many of the complications associated with a wholly implantable device. For example, transcutaneous analyte sensors are generally minimally invasive compared to wholly implantable sensor, and are capable of measuring an analyte concentration for a short period of time (e.g., three days) with similar accuracy as in a wholly implantable sensor.

### SUMMARY OF THE INVENTION

In a first aspect, a transcutaneous analyte sensor is provided, the sensor comprising an elongated flexible portion, wherein the elongated flexible portion has a variable stiffness along at least a portion of its length.

In an embodiment of the first aspect, the sensor comprises at least one wire in a helical configuration, and wherein the variable stiffness is provided by a variable pitch of the helical configuration.

In an embodiment of the first aspect, the sensor comprises at least one wire in a helical configuration, and wherein the variable stiffness is provided by a variable cross-section of the wire.

In an embodiment of the first aspect, the sensor comprises at least one wire, and wherein the variable stiffness is provided by a variable hardness of the wire.

In an embodiment of the first aspect, the variable stiffness of the elongated flexible portion is produced by subjecting the wire to an annealing process.

In an embodiment of the first aspect, the sensor comprises at least one wire, the wire having a variable diameter.

In an embodiment of the first aspect, a distal portion of the sensor is more flexible than a proximal portion of the sensor.

In an embodiment of the first aspect, an intermediate portion of the sensor is more flexible than at least one of a distal portion of the sensor and a proximal portion of the sensor.

In an embodiment of the first aspect, a distal tip of the sensor is stiffer than at least one of an intermediate portion of the sensor and a proximal portion of the sensor.

In a second aspect, a transcutaneous analyte sensor is provided, the sensor comprising a distal portion, an intermediate portion, and a proximal portion, wherein the distal portion is adapted to be inserted through a skin of a host, wherein the proximal portion is adapted to remain substantially external to the host when the distal portion is inserted in the host, and wherein a stiffness of the sensor is variable along a length of the sensor.

In an embodiment of the second aspect, the proximal portion is stiffer than the distal portion.

In an embodiment of the second aspect, the sensor comprises at least one wire in a helical configuration, and wherein a difference in stiffness of the distal portion and the proximal portion is provided by varying a pitch of the helical configuration.

In an embodiment of the second aspect, the sensor comprises at least one wire in a helical configuration, and wherein a difference in flexibility of the distal portion and the proximal portion is provided by a varying a cross-section of the wire.

In an embodiment of the second aspect, the sensor comprises at least one wire, and wherein a difference in flexibility of the distal portion and the proximal portion is provided by a varying a hardness of the wire.

In an embodiment of the second aspect, a variation in stiffness of the elongated flexible portion is produced by subjecting the wire to an annealing process.

In an embodiment of the second aspect, the intermediate portion is more flexible than at least one of the distal portion and the proximal portion.

In an embodiment of the second aspect, the distal portion comprises a distal tip on an end of the sensor that is stiffer a substantial portion of the sensor.

In an embodiment of the second aspect, the intermediate portion is more flexible than at least one of the distal portion and the proximal portion.

In an embodiment of the second aspect, the distal portion comprises a distal tip on an end of the sensor that is stiffer a substantial portion of the sensor.

In a third aspect, a transcutaneous analyte sensor is provided, the sensor comprising an in vivo portion adapted for insertion into a host and an ex vivo portion adapted for operable connection to a device that remains external to the host, wherein the sensor is configured to absorb a relative movement between the ex vivo portion of the sensor and the in vivo portion of the sensor.

In an embodiment of the third aspect, the sensor is configured to absorb a relative movement by a flexibility of at least an intermediate portion located between the in vivo portion and the ex vivo portion.

In an embodiment of the third aspect, the device comprises a housing adapted for mounting on a skin of a host, wherein the housing comprises electrical contacts operably connected to the sensor.

In an embodiment of the third aspect, the ex vivo portion of the sensor is has a preselected stiffness to maintain a stable connection between the sensor and the electrical contacts.

In an embodiment of the third aspect, the in vivo portion of the sensor has a preselected flexibility to minimize mechanical stresses caused by motion of the host.

In an embodiment of the third aspect, a stiffness of the ex vivo portion of the sensor is greater than a stiffness of the in vivo portion of the sensor.

### BRIEF DESCRIPTION OF THE DRAWINGS

FIG. 1A is a perspective view of a transcutaneous sensor assembly.

FIG. 1B is a side cross-sectional view of the transcutaneous sensor assembly of FIG. 1A.

FIG. 2 is a schematic side view of a transcutaneous medical device.

FIG. 3A is a schematic side view of a first transcutaneous medical device having a variable stiffness.

FIG. 3B is a schematic side view of a second transcutaneous medical device having a variable stiffness.

FIG. 3C is a schematic side view of a third transcutaneous medical device having a variable stiffness.

FIGS. 4A to 4D are perspective and side views of a first variable stiffness wire for use with a transcutaneous analyte sensor.

FIGS. 5A and 5B are perspective and cross-sectional views of a second variable stiffness wire for use with a transcutaneous analyte sensor.

FIGS. 6A and 6B are perspective and cross-sectional views of a third variable stiffness wire suitable for use with a transcutaneous analyte sensor.

FIG. 7 is an expanded view of distal and proximal portions of a transcutaneous sensor in one example.

### DETAILED DESCRIPTION OF THE PREFERRED EMBODIMENT

The following description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

### Definitions

In order to facilitate an understanding of the preferred embodiments, a number of terms are defined below.

The term “analyte” as used herein is a broad term and is used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-.beta.hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free .beta.-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, .beta.); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (FHIAA).

The terms “operably connected” and “operably linked” as used herein are broad terms and are used in their ordinary sense, including, without limitation, to refer to one or more components linked to one or more other components. The terms can refer to a mechanical connection, an electrical connection, or a connection that allows transmission of signals between the components. For example, one or more electrodes can be used to detect the amount of analyte in a sample and to convert that information into a signal; the signal can then be transmitted to a circuit. In such an example, the electrode is “operably linked” to the electronic circuitry.

The term “host” as used herein is a broad term and is used in its ordinary sense, including, without limitation, to refer to mammals, particularly humans.

The term “exit-site” as used herein is a broad term and is used in its ordinary sense, including, without limitation, to refer to the area where a medical device (for example, a sensor and/or needle) exits from the host's body.

The phrase “continuous (or continual) analyte sensing” as used herein is a broad term and is used in its ordinary sense, including, without limitation, to refer to the period in which monitoring of analyte concentration is continuously, continually, and or intermittently (regularly or irregularly) performed, for example, about every 5 to 10 minutes.

The term “electrochemically reactive surface” as used herein is a broad term and is used in its ordinary sense, including, without limitation, to refer to the surface of an electrode where an electrochemical reaction takes place. For example, a working electrode measures hydrogen peroxide produced by the enzyme-catalyzed reaction of the analyte detected, which reacts to create an electric current. Glucose analyte can be detected utilizing glucose oxidase, which produces H₂O₂ as a byproduct. H₂O₂ reacts with the surface of the working electrode, producing two protons (2H⁺), two electrons (2e⁻) and one molecule of oxygen (O₂), which produces the electronic current being detected. In the case of the counter electrode, a reducible species, for example, O₂ is reduced at the electrode surface in order to balance the current being generated by the working electrode.

The term “sensing region” as used herein is a broad term and is used in its ordinary sense, including, without limitation, to refer to the region of a monitoring device responsible for the detection of a particular analyte. The sensing region generally comprises a non-conductive body, a working electrode (anode), a reference electrode (optional), and/or a counter electrode (cathode) passing through and secured within the body forming electrochemically reactive surfaces on the body and an electronic connective means at another location on the body, and a multi-domain membrane affixed to the body and covering the electrochemically reactive surface.

The terms “electronic connection” and “electrical connection” as used herein is a broad term and is used in its ordinary sense, including, without limitation, to refer to any electronic connection known to those in the art that can be utilized to interface the sensing region electrodes with the electronic circuitry of a device, such as mechanical (for example, pin and socket) or soldered electronic connections.

The term “domain” as used herein is a broad term and is used in its ordinary sense, including, without limitation, to refer to a region of the membrane system that can be a layer, a uniform or non-uniform gradient (for example, an anisotropic region of a membrane), or a portion of a membrane.

The term “distal to” as used herein is a broad term and is used in its ordinary sense, including, without limitation, the spatial relationship between various elements in comparison to a particular point of reference.

The term “proximal to” as used herein is a broad term and is used in its ordinary sense, including, without limitation, the spatial relationship between various elements in comparison to a particular point of reference.

The terms “in vivo portion” and “distal portion” as used herein are broad terms and are used in their ordinary sense, including, without limitation, to refer to the portion of the device (for example, a sensor) adapted for insertion into and/or existence within a living body of a host.

The terms “ex vivo portion” and “proximal portion” as used herein are broad terms and are used in their ordinary sense, including, without limitation, to refer to the portion of the device (for example, a sensor) adapted to remain and/or exist outside of a living body of a host.

The term “intermediate portion” as used herein is a broad term and is used in its ordinary sense, including, without limitation, to refer to a portion of the device between a distal portion and a proximal portion.

The terms “transdermal” and “transcutaneous” as used herein are broad terms and is used in their ordinary sense, including, without limitation, to refer to extending through the skin of a host. For example, a transdermal analyte sensor is one that extends through the skin of a host.

The term “hardening” as used herein is a broad term and is used in its ordinary sense, including, without limitation, to refer to an increase in hardness of a metal induced by a process such as hammering, rolling, drawing, or the like.

The term “softening” as used herein is a broad term and is used in its ordinary sense, including, without limitation, to refer to an increase in softness of a metal induced by processes such as annealing, tempering, or the like.

The term “tempering” as used herein is a broad term and is used in its ordinary sense, including, without limitation, to refer to the heat-treating of metal alloys, particularly steel, to reduce brittleness and restore ductility.

The term “annealing” as used herein is a broad term and is used in its ordinary sense, including, without limitation, to refer to the treatment of a metal or alloy by heating to a predetermined temperature, holding for a certain time, and then cooling to room temperature to improve ductility and reduce brittleness.

The term “stiff” as used herein is a broad term and is used in its ordinary sense, including, without limitation, to refer to a material not easily bent, lacking in suppleness or responsiveness. In the preferred embodiments, the degree of stiffness can be relative to other portions of the device.

The term “flexible” as used herein is a broad term and is used in its ordinary sense, including, without limitation, to refer to a material that is bendable, pliable, or yielding to influence. In the preferred embodiments, the degree of flexibility can be relative to other portions of the device.

The devices of the preferred embodiments include transdermal medical devices, such as transcutaneous sensor assemblies, with variable stiffness configured along at least a portion of the device. In one aspect of the preferred embodiments, a transcutaneous sensor assembly is provided, including a sensor for sensing an analyte linked to a housing for mounting on the skin of the host. The housing houses an electronics unit associated with the sensor and is adapted for secure adhesion to the host's skin.

### Transcutaneous Sensors

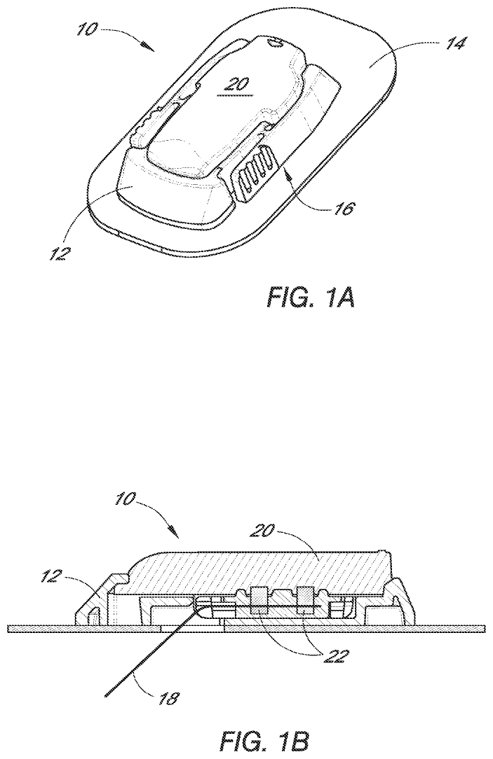

FIGS. 1A and 1B are perspective and side cross-sectional views of a transcutaneous sensor assembly 10 of a preferred embodiment. The sensor system includes a housing 12 and preferably includes an adhesive material 14 on its backside 16 for adhesion to a host's skin. A sensor 18 extends from the housing and is adapted for transdermal insertion through the skin of the host. The sensor portion can be configured for insertion into a variety of in vivo locations, including subcutaneous, venous, or arterial locations, for example. One or more contacts 22 are configured to provide secure electrical contact between sensor 18 and an electronics unit 20. The housing 12 is designed to maintain the integrity of the sensor in the host so as to reduce or eliminate translation of motion between the housing 12, the host, and/or the sensor 18.

In general, the sensor includes at least one electrode configured for measuring an analyte. In one embodiment, the sensor 18 includes at least two electrodes: a working electrode and at least one additional electrode, which can function as a counter and/or reference electrode. Preferably, the working electrode comprises a wire formed from a conductive material, such as platinum, palladium, graphite, gold, carbon, conductive polymer, or the like. In some embodiments, the wire is formed from a bulk material, or alternatively, a composite of two or more metals and/or insulators (e.g., platinum plated steel). The working electrode is configured to measure the concentration of an analyte. The reference electrode, which can function as a reference electrode alone, or as a dual reference and counter electrode, is preferably formed from silver, silver/silver chloride, or the like. In preferred embodiments, the reference electrode is twisted with or around the working electrode; however other configurations for the working electrode and the reference electrode are also possible, for example juxtapositioned, adjacent, coaxial, concentric, interdigitated, spiral-wound, or the like.

In some alternative embodiments, additional electrodes can be included within the assembly. For example, a three-electrode system (working, reference, and counter electrodes) and/or a system including an additional working electrode (which can be used to generate oxygen or can be configured as a baseline subtracting electrode, for example) can be employed. Other sensor/wire/electrode configurations (for example one, two, three, four, or more wires and/or electrode configurations) are also within the scope of the preferred embodiments. For example, U.S. Pat. No. 6,613,379 to Ward et al. describes a bundle of wires around which a counter electrode is deposed and configured for measuring an analyte, and U.S. Pat. No. 6,329,161 to Heller et al. describes a single wire electrode configured for measuring an analyte. Any such configuration adapted for transcutaneous analyte measurement can be configured with a variable stiffness in accordance with the preferred embodiments.

In some embodiments (for example, enzymatic-based sensors), a membrane system is disposed over some or all of the electroactive surfaces of the sensor 18 (working and/or reference electrodes) and provides one or more of the following functions: 1) protection of the exposed electrode surface from the biological environment; 2) diffusion resistance (limitation) of the analyte; 3) a catalyst for enabling an enzymatic reaction; 4) hindering or blocking passage of interfering species; and 5) hydrophilicity at the electrochemically reactive surfaces of the sensor interface, such as is described in co-pending U.S. patent application Ser. No. 11/077,715, filed on Mar. 10, 2005 and entitled “TRANSCUTANEOUS ANALYTE SENSOR”.

The electronics unit 20 can be integral with or removably attached to the housing 12, and includes hardware, firmware and/or software that enable measurement of levels of the analyte via the sensor 18. For example, the electronics unit 20 comprises a potentiostat, a power source for providing power to the sensor, other components useful for signal processing, and preferably an RF module for transmitting data from the electronics unit 20 to a receiver. Electronics can be affixed to a printed circuit board (PCB), or the like, and can take a variety of forms. For example, the electronics can take the form of an integrated circuit (IC), such as an application-specific integrated circuit (ASIC), a microcontroller, or a processor. Preferably, the electronics unit 20 houses the sensor electronics, which comprise systems and methods for processing sensor analyte data. Examples of systems and methods for processing sensor analyte data are described in more detail below and in co-pending U.S. application Ser. No. 10/633,367 filed Aug. 1, 2003 entitled, “SYSTEM AND METHODS FOR PROCESSING ANALYTE SENSOR DATA.”

Co-pending U.S. patent application Ser. No. 11/077,715, filed on Mar. 10, 2005, and entitled, “TRANSCUTANEOUS ANALYTE SENSOR,” describes an embodiment of a transcutaneous analyte sensor that benefits from variable stiffness. Variable stiffness configurations along at least a portion of the device can be employed with devices such as are described in U.S. Pat. No. 6,613,379 to Ward et al., U.S. Pat. No. 6,122,536 to Sun et al., U.S. Pat. No. 6,329,161 to Heller et al., U.S. Pat. No. 6,477,395 to Schulman, and U.S. Pat. No. 4,671,288 to Gough.

### Variable Stiffness

Conventional transcutaneous devices can be subject to motion artifact associated with host movement when the host is using the device. For example, in the example of a transcutaneous analyte sensor such as described above, various movements on the sensor (for example, relative movement within and between the subcutaneous space, dermis, skin, and external portions of the sensor) create stresses on the device, which are known to produce artifacts on the sensor signal.

Accordingly, the design considerations (for example, stress considerations) vary for different sections of a transcutaneous medical device. For example, certain portions of the device can benefit in general from greater flexibility as the portion of the device encounters greater mechanical stresses caused by movement of the tissue within the patient and relative movement between the in vivo and ex vivo portions of the sensor. Additionally or alternatively, certain portions of the device can benefit in general from a stiffer, more robust design to ensure structural integrity and/or reliable electrical connections. Additionally, in some embodiments wherein an insertion device (for example, needle that aids in insertion) is retracted over the device, a stiffer design can enable minimized crimping and/or easy retraction. Thus, by designing greater flexibility into the some portions of the device, the flexibility can compensate for movement and noise associated therewith; and by designing greater stiffness into other portions, column strength (for retraction of the needle over the sensor), electrical connections, and structural integrity can be enhanced.

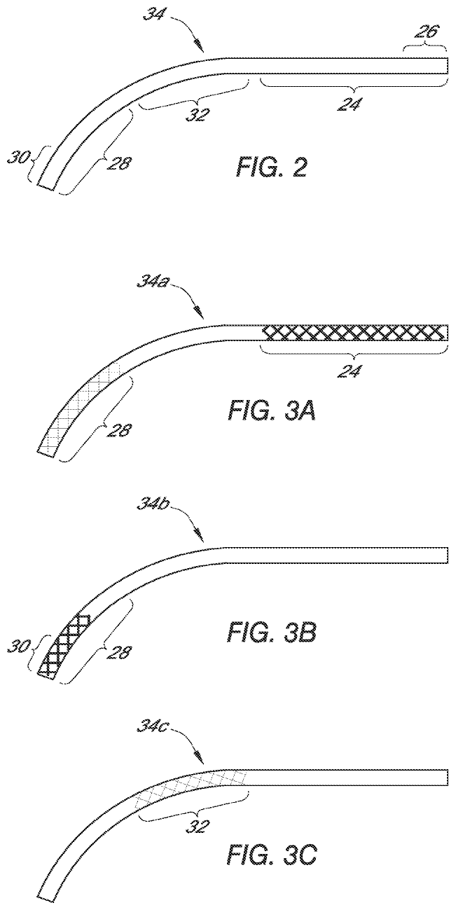

FIG. 2 is a side schematic view of a transcutaneous medical device 34, such as illustrated as the transcutaneous analyte sensor 18 of FIG. 1. In general, a transcutaneous medical device 34, can be divided into three zones, a proximal portion 24 with a proximal tip 26, a distal portion 28 with a distal tip 30, and an intermediate portion 32. The preferred embodiments can employ a variety of configurations that provide variable stiffness along at least a portion of the device in order to overcome disadvantages of conventional transcutaneous devices. Although the following description is focused on an embodiment of a transcutaneous analyte sensor, one skilled in the art can appreciate that the variable stiffness of the preferred embodiments can be implemented with a variety of transcutaneous medical devices.

Generally, the proximal portion 24 is adapted to remain above the host's skin after device insertion and operably connects to a housing ex vivo, for example. The proximal portion 24 typically provides the mechanical and/or electrical connections of the device to housings, electronics, or the like. The proximal portion includes a proximal tip 26 on an end thereof. It is noted that the terms “proximal portion,” “ex vivo portion,” and “proximal tip” do not necessarily imply an exact length or section, rather is generally a section that is more proximal than distal relative to the housing. In some embodiments, the proximal portion (or proximal tip) is stiffer than at least one of the intermediate and distal portions.

Generally, the distal portion 28 of the sensor is adapted for insertion under the host's skin, and is also referred to as the in vivo portion. The distal portion 28 typically provides the device function in vivo, and therefore encounters stresses caused by insertion of the device into the host's tissue and subsequent movement of the tissue of the patient. The distal portion includes a distal tip 30 on an end thereof. It is noted that the terms “distal portion,” “in vivo portion,” and “distal tip” do not necessarily imply an exact length or section, rather is generally a section that is more distal than proximal relative to the housing. In some embodiments, the distal portion is more flexible than at least one of the intermediate and proximal portions. In some embodiments, the distal tip is less flexible than at least one of the remaining (non-tip) distal portion, the intermediate portion, and the proximal portion.

Generally, the intermediate portion 32 is located between the proximal portion 24 and the distal portion and may include portions adapted for insertion or adapted to remain above the skin. The intermediate portion 32 typically provides a transition between the in vivo and ex vivo portions, and can incur stresses caused by relative movement between the in vivo and ex vivo portions of the sensor, for example. It is noted that the term “intermediate portion” does not necessarily imply an exact length or section, rather is generally a section that in-between the proximal and distal portions. In some embodiments, the intermediate portion is more flexible than one or both of the distal and proximal portions.

FIG. 3A is a side schematic view of a transcutaneous medical device 34a in one embodiment adapted for insertion through the skin of a host. In this embodiment, the device 34a is designed with greater flexibility generally in a distal portion 28 (relative to intermediate and/or proximal portions), which is illustrated by light cross-hatching in the distal portion of the device. Stated in another way, the device is designed with a greater stiffness generally in the proximal portion 24 than the intermediate and/or the distal portions, which is illustrated by heavy cross-hatching in the proximal portion 24 of the device. In some embodiments, the intermediate portion includes a flexibility substantially similar to that of the distal portion; in other embodiments, the intermediate portion gradually transitions between the flexibility of the distal portion and the stiffness of the proximal portion. For example, in situations wherein movement of the tissue within the patient and relative movement between the in vivo and ex vivo portions of the device create stresses on the device, greater flexibility in a distal portion (relative to intermediate and/or proximal portions) can provide relief from these mechanical stresses, protecting both the integrity of the sensor and the host tissue. Additionally or alternatively, in situations wherein mechanical and/or electrical connections are required for accurate device function, greater stiffness in the proximal portion 24 (and/or the proximal tip) of the device can increase the stability and reliability of these connections. Thus, the ex-vivo or proximal portion 24 of the sensor is configured for stable connection to the electronics and can additionally be configured to receive an insertion device (such as a needle that aids in sensor insertion) upon retraction from the skin of the host (see co-pending U.S. patent application Ser. No. 11/077,715, filed on Mar. 10, 2005 and entitled “TRANSCUTANEOUS ANALYTE SENSOR”).

FIG. 3B is a side schematic view of a transcutaneous medical device 34b of a preferred embodiment adapted to be inserted through the skin of a host. In this embodiment, the device is designed with an increased stiffness at a distal tip 30 (or a distal portion 28) of the device (relative to intermediate and/or proximal portions) in order to provide increased strength and/or structural integrity to the tip, which is illustrated by heavy cross-hatching. In some situations, the device is inserted into the host such that a tunnel is formed therein. When the device abuts the tunnel end, increased stress to the distal tip can occur. This increased stress can cause the device to bend, resulting in malfunctioning of the device.

In some embodiments, this increased stiffness is designed into the device by creating a greater hardness of the distal tip of the device, for example, by annealing or coating the device. In one embodiment of a transcutaneous analyte sensor as described above with reference to FIG. 1, a higher pitch of the helically wound reference electrode for at least a few windings at a distal end of the reference electrode creates a relative stiffness of the distal portion or tip of the device. It is believed that a stiffer distal portion or tip advantageously provides increased stability, column strength, and proper placement of the device in the host.

FIG. 3C is a side schematic view of a transcutaneous medical device 34c in yet another embodiment adapted to be inserted through the skin of a host. In this embodiment, the device 34c is designed with an increased flexibility at an intermediate portion 32 thereof. Namely, the intermediate portion of the device is designed to absorb shock between the proximal and distal portions, for example, such that movement of the ex vivo portion of the device does not substantially translate to the in vivo portion of the device (and vice versa). In some aspects of this embodiment, the distal portion is designed with a flexibility similar to that of the intermediate portion. In some aspects of this embodiment, the flexibility gradually changes from the distal portion to the proximal portion, including a relatively flexible intermediate portion 32.

In some embodiments, any combination of the above described relatively stiff or flexible portions can be designed into a transcutaneous medical device. In fact, a variety of additional stiff and/or flexible portions can be incorporated into the distal and/or proximal portions of the device without departing from the scope of the preferred embodiments. The flexibility and/or stiffness can be stepped, gradual, or any combination thereof.

The variable stiffness (flexibility) of the preferred embodiments can be provided by a variable pitch of any one or more wires of the device, a variable cross-section of any one or more wires of the device, and/or a variable hardening and/or softening of any one or more wires of the device, for example, as is described in more detail below.

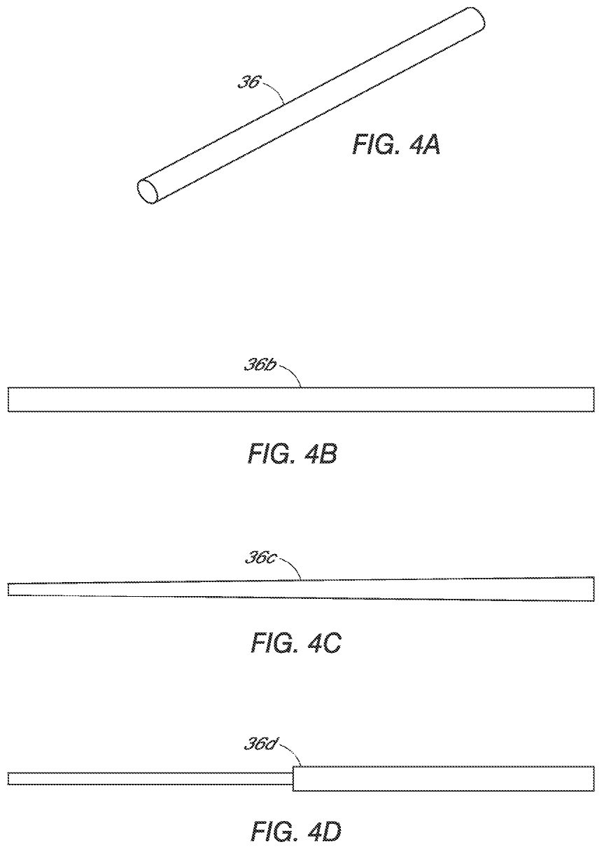

FIGS. 4A to 4D are perspective and side views of a variable stiffness wire used in a transcutaneous medical device, such as an analyte sensor. In FIG. 4A, a wire 36 is shown, which can represent the working electrode or reference electrode of the embodiment described with reference to FIG. 1, for example. Alternatively, the wire 36 can represent one or more wires of a multiple wire sensor (examples of each are described above). The variable stiffness wire described herein can be employed in a transcutaneous medical device to provide variable stiffness along a portion of the length of the device, such as in an analyte sensor.

FIG. 4B is a side view of a variable stiffness wire 36b wherein physical processing of the distal, intermediate, and/or proximal portions of the wire provide for variability of the stiffness of the wire. In some embodiments, some portion (for example, the distal portion) of the wire is softened using a process such as annealing or tempering. In some embodiments, some portion (for example, the proximal portion) of the wire is hardened using a process such as drawing or rolling. In some embodiments, some combination of softening and hardening as described herein are employed to provide variable stiffness of the wire. In the embodiment described with reference to FIG. 1, including a working electrode and a reference electrode, the working electrode can be hardened and/or softened to provide for the variable stiffness of one or more portions of the device, such as is described in more detail elsewhere herein. Another alternative embodiment provides a varying modulus of elasticity of the material to provide the variable stiffness of the preferred embodiments.

FIG. 4C is a side view of an alternative variable stiffness wire 36c, wherein the wire has a gradually increasing or decreasing diameter along its length. The variability in diameter can be produced by physical or chemical processes, for example, by grinding, machining, rolling, pulling, etching, drawing, swaging, or the like. In this way, a transcutaneous analyte sensor, or other transcutaneous medical device, can be produced having a variable stiffness. In the embodiment described with reference to FIG. 1, for example, including a working electrode and a reference electrode, the working electrode can be formed with a variable diameter to provide for the variable stiffness of one or more portions of the device, such as described in more detail elsewhere herein.

FIG. 4D is a side view of another alternative variable stiffness wire 36d, wherein the wire is step increased or decreased to provide two (or more) different flexibilities of the wire. The wire can be stepped by physical or chemical processes known in the art, such as described with reference to FIG. 4C. In this way, a transcutaneous analyte sensor, or other transcutaneous medical device, can be produced with a variable stiffness. A noted advantage of the smaller diameter configurations of FIGS. 4C and 4D include reduced sizing of the in vivo portion of the device, which is believed to be more comfortable for the patient and to induce less invasive trauma around the device, thereby providing an optimized device design.

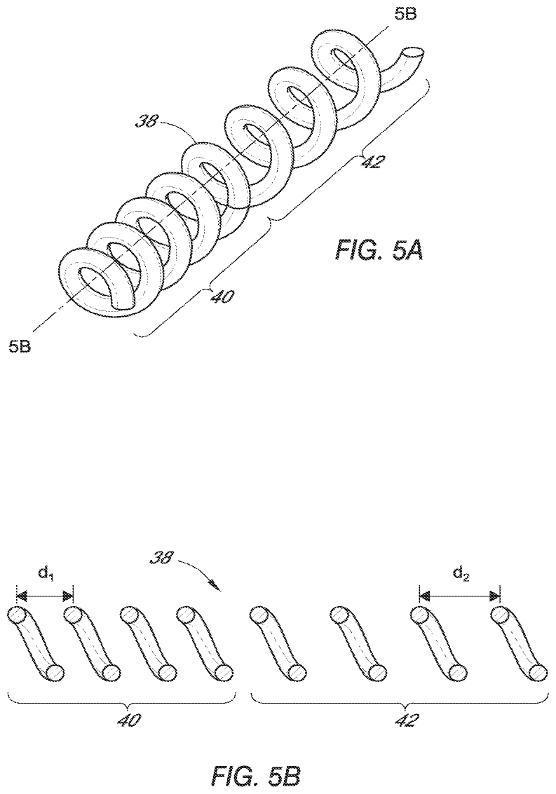

FIGS. 5A and 5B are perspective and cross-sectional views of a variable stiffness wire 38 in an alternative embodiment representing any one or more wires associated with a transcutaneous medical device, such as an analyte sensor. For example, the wire 38 can represent the reference electrode of the embodiment described with reference to FIG. 1. Alternatively, the wire 38 can represent the wire of a single or multiple wire sensor (examples of each are described above).

In this embodiment, two distinct portions 40, 42 are shown with first and second pitches; however, the illustration is not meant to be limiting and the variable pitch can include any number of gradual portions, stepped portions, or the like. Additionally, the variable pitch and/or helical configuration can be provided on only a portion of the wire or on the entire length of the wire, and can include any number of pitch changes. In this embodiment, a first portion 40 is wound to have relatively closely spaced coils, namely, a high helix pitch, whereas a second 42 portion is not subjected to high stress levels and can include coils wound with a lower helix pitch. The helix pitch is defined as the number of coils of the wire core per unit length of the device, or the distance between the coils.

FIG. 5B is a cross-sectional view along line B-B of the device of FIG. 5A, illustrating a first distance d₁ between the coils in the first portion 40 and a second distance d₂ between the coils in the second portion 42, wherein d₂ is greater than d₁. Thus, the wire has a variable stiffness attributable to the varying helix pitch over the length of the sensor. In this way, portions of a device having wire with a low helix pitch are designed with greater flexibility and are more able to handle the stresses associated with motion of the sensor while portions of the sensor having wire with a high helix pitch are designed with more stiffness and provide more stability for the sensor in the housing. Any portions (proximal, intermediate, and/or distal portions (or tips)) can be designed with a variable pitch to impart variable stiffness.

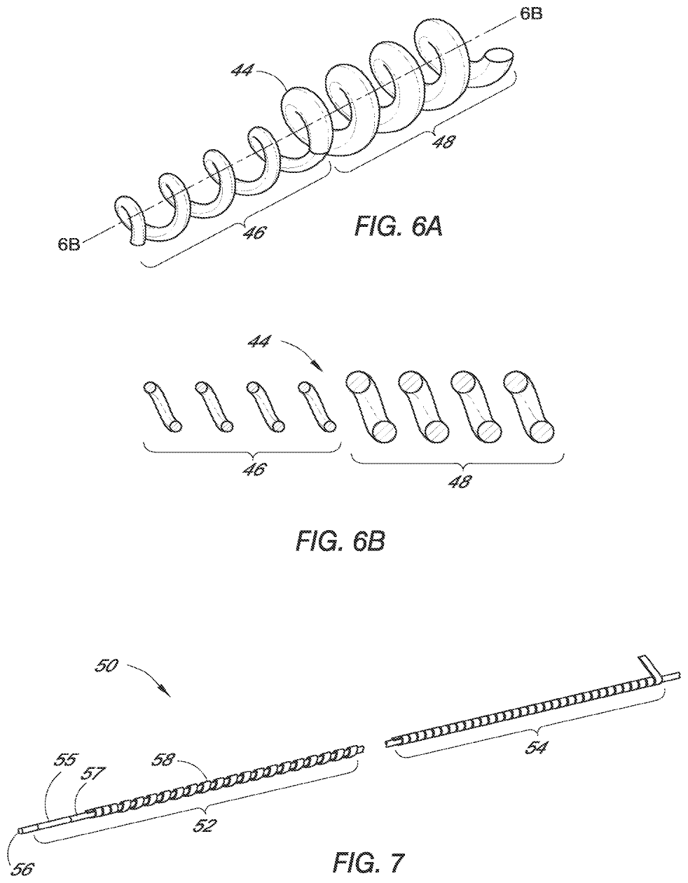

FIGS. 6A and 6B are perspective and longitudinal views of a variable stiffness wire 44 in yet another alternative embodiment representing any one or more wires associated with a transcutaneous medical device, such as an analyte sensor. For example, the wire 44 can be the reference electrode of the embodiment described with reference to FIG. 1. Alternatively, the wire 44 can be a working electrode, and/or one or more wires of a multiple wire sensor (examples of each are described above).

In this embodiment, two distinct portions 46, 48 are shown with first and second wire diameters that provide a variable cross-section; however, the illustration is not meant to be limiting and the variable cross-section can be gradual, stepped, or the like. Additionally, the variable cross-section and/or helical configuration can be provided on only a portion of the wire or on the entire length of the wire, and can include any number of cross-section changes. In this embodiment, the helically wound wire is designed with a variable cross-sectional area over the length of the sensor from a small cross-sectional area in the first portion 46 to a larger cross-sectional area in the second portion 48.

FIG. 6B is a cross-sectional view along line B-B of the device of FIG. 6A, revealing cross-sectional information about one or more wires that make up the coil, including a first cross-section x, of the wire in the first portion 20 and a second cross-section x₂ of the wire in the second portion 48, wherein x₂ is greater than x₁. Thus, the device of this embodiment has a variable stiffness attributable to the varying cross-section over the length of the sensor. In this way, first portion 46 has a smaller cross-sectional area and is therefore more flexible and capable of withstanding the stresses associated with patient movement, for example; while the second portion 48 has a larger cross-sectional area and is stiffer and provides more stability and column strength desirable for mechanical and electrical connections, for example.

The transcutaneous analyte sensor of FIG. 1 includes a helical configuration. The helical surface topography of the reference electrode surrounding the working electrode not only provides electrochemical functionality, but can also provide anchoring within the host tissue. The device preferably remains substantially stationary within the tissue of the host, such that migration or motion of the sensor with respect to the surrounding tissue is minimized. Migration or motion can cause inflammation at the sensor implant site due to irritation and can also cause noise on the sensor signal due to motion-related artifact, for example. Therefore, it can be advantageous to provide an anchoring mechanism that provides support for the sensor in vivo portion to avoid or minimize the above-mentioned problems. Combining advantageous sensor geometry with advantageous anchoring minimizes additional parts in the device, and allows for an optimally small or low profile design of the sensor. Additionally or alternatively, anchoring can be provided by prongs, spines, barbs, wings, hooks, rough surface topography, gradually changing diameter, or the like, which can be used alone or in combination with the helical surface topography to stabilize the sensor within the subcutaneous tissue.

### EXAMPLE

FIG. 7 is an expanded view of distal and proximal portions of a transcutaneous sensor 50 in one example. FIG. 7 illustrates a sensor 50 broken away between its distal portion 52 and proximal portion 54, representing any length or configuration there between. In the illustrated embodiment, the sensor 50 includes two electrodes: a working electrode 56 and one additional electrode, which can function as a counter and/or reference electrode, hereinafter referred to as the reference electrode 58. Each electrode is formed from a fine wire with a diameter of approximately 0.0045 inches.

The working electrode 56 comprises a platinum wire and is configured and arranged to measure the concentration of an analyte. In this example of an enzymatic electrochemical sensor, the working electrode measures the hydrogen peroxide produced by an enzyme catalyzed reaction of the analyte being detected and creates a measurable electronic current (for example, detection of glucose utilizing glucose oxidase produces H₂O₂ peroxide as a by product, H₂O₂ reacts with the surface of the working electrode producing two protons (2H⁺), two electrons (2e⁻) and one molecule of oxygen (O₂) which produces the electronic current being detected).

The working electrode 56 is covered with an insulator 57, e.g., Parylene, which is vapor-deposited on the working electrode. Parylene is an advantageous conformal coating because of its strength, lubricity, and electrical insulation properties; however, a variety of other insulating materials can also be used, for example, fluorinated polymers, polyethyleneterephthalate, polyurethane, polyimide, or the like. The reference electrode 58, which can function as a counter electrode alone, or as a dual reference and counter electrode, is preferably silver or a silver-containing material. In this example, the reference electrode 58 is helically twisted around the working electrode 56. A window 55 is formed on the insulating material to expose an electroactive surface of the working electrode 56. Other methods and configurations for exposing electroactive surfaces can also be employed.

In this example, the reference electrode 58 is wound with a variable pitch that creates a variable stiffness along the length of the sensor 50. Namely, the sensor 50 is designed with a greater stiffness generally in the proximal portion 54 than the intermediate and/or the distal portions 52. However, an increased stiffness of a section of the distal portion 52, shown adjacent to the window 55 wherein the reference electrode 58 includes a higher helix pitch for a few windings, provides increased strength in a high stress location, without inhibiting the overall flexibility of the distal portion 52. It is believed that in situations wherein movement of the tissue within the patient and relative movement between the in vivo and ex vivo portions of the device create stresses on the device, greater flexibility in a distal portion (and optionally in the intermediate portion relative to the proximal portion) can provide relief from these mechanical stresses, protecting both the integrity of the sensor and the host tissue. Additionally or alternatively, in situations wherein mechanical and/or electrical connections are employed for accurate function, greater stiffness in the proximal portion (and/or the proximal tip) of the device can increase the stability and reliability of these connections. Additionally, this exemplary configuration is advantageous for the reasons described above, and further provides an enhanced mechanical stability by the distribution of forces of the helical wire along the straight wire.

Methods and devices that are suitable for use in conjunction with aspects of the preferred embodiments are disclosed in U.S. Pat. No. 4,994,167 issued Feb. 19, 1991 and entitled “BIOLOGICAL FLUID MEASURING DEVICE”; U.S. Pat. No. 4,757,022 issued Feb. Jul. 12, 1988 and entitled “BIOLOGICAL FLUID MEASURING DEVICE”; U.S. Pat. No. 6,001,067 issued Feb. Dec. 14, 1999 and entitled “DEVICE AND METHOD FOR DETERMINING ANALYTE LEVELS”; U.S. Pat. No. 6,741,877 issued Feb. May 25, 2004 and entitled “DEVICE AND METHOD FOR DETERMINING ANALYTE LEVELS”; U.S. Pat. No. 6,702,857 issued Feb. Mar. 9, 2004 and entitled “MEMBRANE FOR USE WITH IMPLANTABLE DEVICES”; and U.S. Pat. No. 6,558,321 issued Feb. May 6, 2003 and entitled “SYSTEMS AND METHODS FOR REMOTE MONITORING AND MODULATION OF MEDICAL DEVICES.” Methods and devices that are suitable for use in conjunction with aspects of the preferred embodiments are disclosed in co-pending U.S. application Ser. No. 10/991,353 filed Nov. 16, 2004 and entitled “AFFINITY DOMAIN FOR ANALYTE SENSOR”; U.S. application Ser. No. 11/055,779 filed Feb. 9, 2005 and entitled “BIOINTERFACE WITH MACRO-AND-MICRO-ARCHITECTURE”; U.S. application Ser. No. 11/004,561 filed Dec. 3, 2004 and entitled “CALIBRATION TECHNIQUES FOR A CONTINUOUS ANALYTE SENSOR”; U.S. application Ser. No. 11/034,343 filed Jan. 11, 2005 and entitled “COMPOSITE MATERIAL FOR IMPLANTABLE DEVICE”; U.S. application Ser. No. 09/447,227 filed Nov. 22, 1999 and entitled “DEVICE AND METHOD FOR DETERMINING ANALYTE LEVELS”; U.S. application Ser. No. 11/021,046 filed Dec. 22, 2004 and entitled “DEVICE AND METHOD FOR DETERMINING ANALYTE LEVELS”; U.S. application Ser. No. 09/916,858 filed Jul. 27, 2001 and entitled “DEVICE AND METHOD FOR DETERMINING ANALYTE LEVELS”; U.S. application Ser. No. 11/039,269 filed Jan. 19, 2005 and entitled “DEVICE AND METHOD FOR DETERMINING ANALYTE LEVELS”; U.S. application Ser. No. 10/897,377 filed Jul. 21, 2004 and entitled “ELECTROCHEMICAL SENSORS INCLUDING ELECTRODE SYSTEMS WITH INCREASED OXYGEN GENERATION”; U.S. application Ser. No. 10/897,312 filed Jul. 21, 2004 and entitled “ELECTRODE SYSTEMS FOR ELECTROCHEMICAL SENSORS”; U.S. application Ser. No. 10/838,912 filed May 3, 2004 and entitled “IMPLANTABLE ANALYTE SENSOR”; U.S. application Ser. No. 10/838,909 filed May 3, 2004 and entitled “IMPLANTABLE ANALYTE SENSOR”; U.S. application Ser. No. 10/838,658 filed May 3, 2004 and entitled “IMPLANTABLE ANALYTE SENSOR”; U.S. application Ser. No. 11/034,344 filed Jan. 11, 2005 and entitled “IMPLANTABLE DEVICE WITH IMPROVED RADIO FREQUENCY CAPABILITIES”; U.S. application Ser. No. 10/896,772 filed Jul. 21, 2004 and entitled “INCREASING BIAS FOR OXYGEN PRODUCTION IN AN ELECTRODE SYSTEM”; U.S. application Ser. No. 10/789,359 filed Feb. 26, 2004 and entitled “INTEGRATED DELIVERY DEVICE FOR CONTINUOUS GLUCOSE SENSOR”; U.S. application Ser. No. 10/991,966 filed Nov. 17, 2004 and entitled “INTEGRATED RECEIVER FOR CONTINUOUS ANALYTE SENSOR”; U.S. application Ser. No. 10/646,333 filed Aug. 22, 2003 and entitled “OPTIMIZED SENSOR GEOMETRY FOR AN IMPLANTABLE GLUCOSE SENSOR”; U.S. application Ser. No. 10/896,639 filed Jul. 21, 2004 and entitled “OXYGEN ENHANCING MEMBRANE SYSTEMS FOR IMPLANTABLE DEVICES”; U.S. application Ser. No. 10/647,065 filed Aug. 22, 2003 and entitled “POROUS MEMBRANES FOR USE WITH IMPLANTABLE DEVICES”; U.S. application Ser. No. 10/896,637 filed Jul. 21, 2004 and entitled “ROLLED ELECTRODE ARRAY AND ITS METHOD FOR MANUFACTURE”; U.S. application Ser. No. 09/916,711 filed Jul. 27, 2001 and entitled “SENSOR HEAD FOR USE WITH IMPLANTABLE DEVICE”; U.S. application Ser. No. 11/021,162 filed Dec. 22, 2004 and entitled “SENSOR HEAD FOR USE WITH IMPLANTABLE DEVICES”; U.S. application Ser. No. 11/007,920 filed Dec. 8, 2004 and entitled “SIGNAL PROCESSING FOR CONTINUOUS ANALYTE SENSOR”; U.S. application Ser. No. 10/695,636 filed Oct. 28, 2003 and entitled “SILICONE COMPOSITION FOR BIOCOMPATIBLE MEMBRANE”; U.S. application Ser. No. 11/038,340 filed Jan. 18, 2005 and entitled “SYSTEM AND METHODS FOR PROCESSING ANALYTE SENSOR DATA”; U.S. application Ser. No. 11/007,635 filed Dec. 7, 2004 and entitled “SYSTEMS AND METHODS FOR IMPROVING ELECTROCHEMICAL ANALYTE SENSORS”; U.S. application Ser. No. 10/885,476 filed Jul. 6, 2004 and entitled “SYSTEMS AND METHODS FOR MANUFACTURE OF AN ANALYTE-MEASURING DEVICE INCLUDING A MEMBRANE SYSTEM”; U.S. application Ser. No. 10/648,849 filed Aug. 22, 2003 and entitled “SYSTEMS AND METHODS FOR REPLACING SIGNAL ARTIFACTS IN A GLUCOSE SENSOR DATA STREAM”; U.S. application Ser. No. 10/153,356 filed May 22, 2002 and entitled “TECHNIQUES TO IMPROVE POLYURETHANE MEMBRANES FOR IMPLANTABLE GLUCOSE SENSORS”; U.S. application Ser. No. 10/846,150 filed May 14, 2004 and entitled “ANALYTE MEASURING DEVICE”; U.S. application Ser. No. 10/842,716 filed May 10, 2004 and entitled “BIOINTERFACE MEMBRANES INCORPORATING BIOACTIVE AGENTS”; U.S. application Ser. No. 10/657,843 filed Sep. 9, 2003 and entitled “DEVICE AND METHOD FOR DETERMINING ANALYTE LEVELS”; U.S. application Ser. No. 10/768,889 filed Jan. 29, 2004 and entitled “MEMBRANE FOR USE WITH IMPLANTABLE DEVICES”; U.S. application Ser. No. 10/633,367 filed Aug. 1, 2003 and entitled “SYSTEM AND METHODS FOR PROCESSING ANALYTE SENSOR DATA”; U.S. application Ser. No. 10/632,537 filed Aug. 1, 2003 and entitled “SYSTEM AND METHODS FOR PROCESSING ANALYTE SENSOR DATA”; U.S. application Ser. No. 10/633,404 filed Aug. 1, 2003 and entitled “SYSTEM AND METHODS FOR PROCESSING ANALYTE SENSOR DATA”; U.S. application Ser. No. 10/633,329 filed Aug. 1, 2003 and entitled “SYSTEM AND METHODS FOR PROCESSING ANALYTE SENSOR DATA”; and U.S. application Ser. No. 60/660,743, filed on Mar. 10, 2005 and entitled “SYSTEMS AND METHODS FOR PROCESSING ANALYTE SENSOR DATA FOR SENSOR CALIBRATION.”

All references cited herein, including but not limited to published and unpublished applications, patents, and literature references, and also including but not limited to the references listed in the Appendix, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. A transcutaneous analyte sensor, the sensor comprising an elongated flexible portion, wherein the elongated flexible portion has a variable stiffness along at least a portion of its length.

2. The sensor of claim 1, wherein the sensor comprises at least one wire in a helical configuration, and wherein the variable stiffness is provided by a variable pitch of the helical configuration.

3. The sensor of claim 1, wherein the sensor comprises at least one wire in a helical configuration, and wherein the variable stiffness is provided by a variable cross-section of the wire.

4. The sensor of claim 1, wherein the sensor comprises at least one wire, and wherein the variable stiffness is provided by a variable hardness of the wire.

5. The sensor of claim 4, wherein the variable stiffness of the elongated flexible portion is produced by subjecting the wire to an annealing process.

6. The sensor of claim 1, wherein the sensor comprises at least one wire, the wire having a variable diameter.

7. The sensor of claim 1, wherein a distal portion of the sensor is more flexible than a proximal portion of the sensor.

8. The sensor of claim 1, wherein an intermediate portion of the sensor is more flexible than at least one of a distal portion of the sensor and a proximal portion of the sensor.

9. The sensor of claim 1, wherein a distal tip of the sensor is stiffer than at least one of an intermediate portion of the sensor and a proximal portion of the sensor.

10. A transcutaneous analyte sensor, the sensor comprising a distal portion, an intermediate portion, and a proximal portion, wherein the distal portion is adapted to be inserted through a skin of a host, wherein the proximal portion is adapted to remain substantially external to the host when the distal portion is inserted in the host, and wherein a stiffness of the sensor is variable along a length of the sensor.

11. The sensor of claim 10, wherein the proximal portion is stiffer than the distal portion.

12. The sensor of claim 11, wherein the sensor comprises at least one wire in a helical configuration, and wherein a difference in stiffness of the distal portion and the proximal portion is provided by varying a pitch of the helical configuration.

13. The sensor of claim 11, wherein the sensor comprises at least one wire in a helical configuration, and wherein a difference in flexibility of the distal portion and the proximal portion is provided by a varying a cross-section of the wire.

14. The sensor of claim 11, wherein the sensor comprises at least one wire, and wherein a difference in flexibility of the distal portion and the proximal portion is provided by a varying a hardness of the wire.

15. The sensor of 14, wherein a variation in stiffness of the elongated flexible portion is produced by subjecting the wire to an annealing process.

16. The sensor of claim 11, wherein the intermediate portion is more flexible than at least one of the distal portion and the proximal portion.

17. The sensor of claim 16, wherein the distal portion comprises a distal tip on an end of the sensor that is stiffer a substantial portion of the sensor.

18. The sensor of claim 10, wherein the intermediate portion is more flexible than at least one of the distal portion and the proximal portion.

19. The sensor of claim 18, wherein the distal portion comprises a distal tip on an end of the sensor that is stiffer a substantial portion of the sensor.

20. A transcutaneous analyte sensor, the sensor comprising an in vivo portion adapted for insertion into a host and an ex vivo portion adapted for operable connection to a device that remains external to the host, wherein the sensor is configured to absorb a relative movement between the ex vivo portion of the sensor and the in vivo portion of the sensor.
